(19) United States
(12) Patent Application Publication
(10) Pub. No.: US 2019/0029585 A1
(43) Pub. Date: Jan. 31, 2019

(54) INTERACTIVE SYSTEM AND METHOD FOR THE DIAGNOSIS AND TREATMENT OF SOCIAL COMMUNICATION OR ATTENTION DISORDERS IN INFANTS AND CHILDREN

(71) Applicant: BAR-ILAN UNIVERSITY, Ramat Gan (IL)

(72) Inventors: Ronny GEVA, Tel Aviv (IL); Michal ZIVAN, Binyamina (IL)

(21) Appl. No.: 16/048,499

(22) Filed: Jul. 30, 2018

Related U.S. Application Data
(60) Provisional application No. 62/538,841, filed on Jul. 31, 2017.

(51) Int. Cl. A61B 5/16; G06F 3/01

(52) U.S. Cl. CPC A61B 5/168; A61M 2021/005; G06F 3/013; A61B 5/163

(57) ABSTRACT

The present invention relates to an interactive gaze contingent system and to methods of for evaluating and/or diagnosing and/or treating a social communication disorder or an attention disorder in infants and children at risk or diagnosed with social communication disorders or attention disorders.

The patent or application file contains at least one drawing executed in color. Copies of this patent or patent application publication with color drawing(s) will be provided by the Office upon request and payment of the necessary fee.

### FIELD OF THE INVENTION

The present invention relates to an interactive gaze contingent system and to methods of diagnosis and treatment of infants and children at risk or diagnosed with social communication disorders or attention disorders.

### BACKGROUND OF THE INVENTION

Over the last two decades a dramatic increase in the prevalence of social communication disorders was reported, 1 of every 68 children was diagnosed with autism spectrum disorder (ASD) in the U.S in 2012, according to the centers for disease control and prevention (CDC) [Available from: https://www.cdc.gov/ncbddd/autism/data.html]. Further, the age for diagnosis is according to available means, at least 2-3 years of age, which does not enable early intervention. For ASD, for example, which includes a wide range of neurodevelopmental disorders, characterized by social and communicative deficits, the median age of diagnosis is 3 years and 10 months [Identified prevalence of autism spectrum disorders, 2012, Available from: https://www.cdc.gov/ncbddd/autism/data.html], yet it is clear that early intervention can bring better future functional outcome [Dawson, G., Early behavioral intervention, brain plasticity, and the prevention of autism spectrum disorder. Development and Psychopathology, 2008. 20(03): p. 775-803].

According to the American Speech-Language-Hearing Association (ASHA), social communication disorders include problems with social interaction (e.g., speech style and context, rules for linguistic politeness), social cognition (e.g., emotional competence, understanding emotions of self and others), and pragmatics (e.g., communicative intentions, body language, eye contact). A social communication disorder may be a distinct diagnosis or may occur within the context of other conditions, such as autism spectrum disorder (ASD), specific language impairment (SLI), learning disabilities (LD), language learning disabilities (LLD), intellectual disabilities (ID), developmental disabilities (DD), attention deficit hyperactivity disorder (ADHD), and traumatic brain injury (TBI). Other conditions (e.g., psychological/emotional disorders and hearing loss) may also impact social communication skills. In the case of ASD, social communication problems are a defining feature along with restricted, repetitive patterns of behavior.

Several study groups have begun prospective studies with infants at higher risk to develop ASD, in order to understand the diverted developmental course of individuals with ASD. Mix evidence was found regarding the early symptoms emerging during the first year of life in infants later diagnosed with ASD [Elsabbagh, M. and M. H. Johnson, Autism and the Social Brain: The First-Year Puzzle. Biological psychiatry. 80(2): p. 94-99].

Early behavioral markers in children later diagnosed with social communication disorders include: difficulties in gaze orienting, attention and exploration, preference of non-social stimuli oversocial stimuli (faces) and atypical and reduced eye contact. However, standard intervention usually starts only after the age of 3 years (and sometimes much later) and includes speech therapy/emotional therapy. There are different populations of infants at higher risk to develop ASD and its broader phenotype. Two main risk factors are genetic risk and prematurity birth. The two main populations of infants at risk to develop ASD, siblings of children that have been diagnosed on the autistic spectrum and infants that were born preterm, are fairly prevalent in the general population. ASD has a strong genetic basis and siblings of children diagnosed on the autistic spectrum have an 18.7% chance [Ozonoff, S., et al., Recurrence Risk forAutismSpectrum Disorders: A Baby Siblings Research Consortium Study. Pediatrics, 2011. 128(3): p. e488-e495. Prematurity birth holds another major risk factor for the development of ASD. Studies with extremely low birth ex-preterms, report that 25% of ex-preterm infants (birth weight<1500 gram) had a positive screen for early autistic features at a mean corrected age of 22 month [Pinelli, J. and L. Zwaigenbaum., Chorioamnionitis, gestational age, male sex, birth weight, and illness severity predicted positive autism screening scores in very-low-birth-weight preterm infants. Evidence Based Nursing, 2008. 11(4): 122; Limperopoulos, C., H. Bassan, et al., Positive Screening for Autism in Ex-preterm Infants: Prevalence and Risk Factors. Pediatrics, 2008. 121(4):758-765].

Eye tracking is a useful tool for the study of gaze behavior and preference at infancy. Prospective eye tracking research on infants at high risk has emerged in the last few years especially with siblings cohorts, with mixed evidence regarding atypical social behavior in the first year of infants later diagnosed with ASD.

To date, an efficient method is lacking for the early diagnosis of social communication disorders or attention, as are interactive tools for treating infants and children at risk or diagnosed with social communication disorders or attention disorders.

Current methods of diagnosis of social communication disorders rely on basic communication skills of the children. Moreover, the diagnosis and treatment mainly consist of playful interaction of the child with a professional. Indeed, there are very few computer-based programs in this field, and these are unable of receiving input from the subject without his active use of an input device (such as a mouse, keyboard or touch screen etc.). As such, these programs are limited to those subjects which are capable of using an input device and can understand instructions.

Newly diagnosed toddlers and their parents often need to wait several months before starting therapy, due to the overload in the developmental centers, and as a consequence these children do not receive adequate treatment.

It would therefore be extremely important, and this is an object of the present invention, to provide an interactive diagnostic and therapeutic tool for infants and children at risk or diagnosed with social communication disorders and attention disorders.

It is another object of the invention to provide solutions to the unmet need of early diagnosis of social communication disorders and attention disorders in infants, and for the diagnosis of low-functioning older children.

It is a further object of the invention to provide such methods and systems which do not require active use of an input device by the infant or child.

It is yet another object of the invention to provide a method for the early treatment of children at risk or diagnosed with social communication disorders and attention disorders, using individually-tailored intervention.

It is still another object of the invention to provide a low-cost solution that may be utilized in a clinic or even in a home setting.

Another object of the invention is the provision of an interactive gaze contingent system for the diagnosis and treatment of infants and children at risk or diagnosed with social communication disorders and attention disorders.

The above and other purposes and advantages of the invention will become apparent as the description proceeds.

### SUMMARY OF THE INVENTION

The present invention provides an interactive gaze contingent system for evaluating and/or diagnosing and/or treating a social communication disorder or an attention disorder in a subject, comprising: a display; an eye tracker component; and a processing component operatively connected to the display and eye tracker component. The processing component comprises circuitry adapted to initiate and to discontinue activities displayed on the display as a result of input received from the eye tracker component.

According to some embodiments, the eye tracker of the interactive gaze contingent system is configured to obtain data from the subject's gaze position and to communicate it to the processing component. The circuitry associated with the processing component is configured to change the image or sequence of images displayed on the display as a function of the subject's gaze position.

According to one embodiment, the present invention provides a method for the diagnosis and/or evaluation and/or treatment of a disorder in a subject comprising placing said subject in proximity to a gaze contingent system, displaying a battery of tasks on the display of said system; obtaining data from the subject's gaze position and communicating the data to the processing component; changing the matter displayed on the display as a function of the subject's gaze position; and analyzing the data obtained from the subject's gaze response to said tasks. According to some embodiments, the method includes comparing data from the subject's gaze response with gaze patterns of healthy subjects.

According to some embodiments, the method of treatment of a disorder in a subject further comprises modifying the task displayed on the display for encouraging a social behavior.

According to some embodiments, the battery of tasks comprises social and visual attention tasks which may be images and clips that are displayed on the display.

According to some embodiments of the invention, the disorder is a social communication disorder or an attention disorder which may be associated with autism spectrum disorder (ASD), specific language impairment (SLI), learning disabilities (LD), language learning disabilities (LLD), intellectual disabilities (ID), developmental disabilities (DD), attention deficit hyperactivity disorder (ADHD), attention deficit disorder (ADD) and traumatic brain injury (TBI).

In some embodiments of the present invention, the subject diagnosed/treated is a child, in some embodiments the subjects is an infant, who may be in some cases less than 2 years old and even a few months old.

### BRIEF DESCRIPTION OF THE DRAWINGS

FIG. 1 shows one embodiment of the gaze contingent system of the invention;

FIG. 2 is a screen shot of an illustrative direct gaze Vs. averted gaze task;

FIG. 3 shows the average total time at direct gaze Vs. averted gaze in preterm, siblings and control groups; and

FIG. 4 shows the average total time at direct gaze Vs. averted gaze in non-concern group compared to concern group.

FIG. 5 shows the total average time (in percentage) at simple stimulus Vs. complex stimulus in siblings, preterm and control groups.

FIG. 6A shows the average first fixation time at simple stimulus Vs. complex stimulus in siblings, preterm and control groups.

FIG. 6B shows average fixation time at the at simple stimulus Vs. complex stimulus in siblings, preterm and control groups.

### DETAILED DESCRIPTION OF EMBODIMENTS OF THE INVENTION

Infants at high risk of developing a social communication disorder present behaviors such as lack of preference for eye contact already during the first year of life. It was found that the infants' preference to direct eye gaze over averted eye gaze at 9 months of age, studied using a gaze contingent system, provides an effective diagnostic and treatment tool.

Gaze contingent methodology allows infants to control the task with their own gaze at their own pace, including infants with marked developmental risk. Unlike other experimental systems for infants, this system has a minimal “forced exposure” component and thus enables a “cleaner” reflection of the child's gaze preferences.

Gaze contingent paradigm is an interactive tool that influences the task's progress depending on the subject interest focus [Lloyd-Fox, S., et al., Reduced neural sensitivity to social stimuli in infants at risk for autism. Proceedings of the Royal Society of London B: Biological Sciences, 2013. 280(1758)]. This paradigm is especially useful for infants that are not capable of actively using a computer input device (such as a mouse, keyboard or touch screen etc.). Using this system, the subject can actively control the task progress or choose according to his own preference and pace. Using this paradigm allows studying the gaze behavior of infants during a social task that is self-operated by their gaze. This approach allows recording of the infant's own and unique gaze pattern spontaneously, without presenting them with a forced choice or meeting a criterion for minimum exposure. Gaze contingency enables the evaluation of gaze regulation as a function of self-motivation in a natural and realistic fashion.

The present invention relates, among other things, to a system and a plurality of methods of utilizing a gaze contingent paradigm for diagnosis and therapy of infants and children at risk or diagnosed with a social communication disorder or attention disorder. The system assists in providing early diagnosis. The system further assists in early intervention, but is also useful for all age levels, as an educational tool for both behavioral and cognitive therapy for a child. According to the present invention the infants can operate live videos clips by themselves, by simply fixating at them.

The gaze operating system is very intuitive and enables to create a realistic and adjusted environment that is ideal for the treatment of infants and children with developmental deficits such as social communication disorders and attention disorders. The low cost of eye-tracking permits to provide an easy-to-use treatment tool at home for daily use. The treatment may have a profound impact on the first years of life and will lead to a more promising long-term functional outcome for these subjects.

The system of the present invention is low-cost, compact and easy to use. The present invention may be utilized in a pediatrician's office, in a medical facility with a therapist and can even be utilized in a home setting.

As used herein, the terms “gaze contingent system” and “gaze contingent paradigm” refer to techniques allowing a computer screen display to change in function, depending on where the viewer is looking. The term also encompasses an interactive mode where the system responds to the observer's actions and interacts with him.

As used herein, the term “subject” refers to infants, children, adolescents, adults, elderly, disabled, or veterans, in the context of this disclosure, the terms child and children are used as examples of subjects and refer to children with or suspected of being afflicted with social communication disorders and attention disorders.

As used herein, the term “diagnosis” refers to detecting and identifying a disease/disorder in a subject. The term may also encompass assessing or evaluating the disease/disorder status (severity, classification, progression, regression, stabilization, response to treatment, etc.) in a patient. The diagnosis may include a prognosis of the disease/disorder in the subject.

As used herein, the term “social communication disorders” includes problems with social interaction (e.g., speech style and context, rules for linguistic politeness), social cognition (e.g., emotional competence, understanding emotions of self and others), and pragmatics (e.g., communicative intentions, body language, eye contact). A social communication disorder may be a distinct diagnosis or may occur within the context of other conditions, such as autism spectrum disorder (ASD), specific language impairment (SLI), learning disabilities (LD), language learning disabilities (LLD), intellectual disabilities (ID), developmental disabilities (DD), attention deficit hyperactivity disorder (ADHD), and traumatic brain injury (TBI). Other conditions (e.g., psychological/emotional disorders and hearing loss) may also impact social communication skills.

As used herein, the term “attention disorder” relates to disorders that are marked especially by persistent symptoms of inattention (such as distractibility, forgetfulness, or disorganization) or by symptoms of hyperactivity and impulsivity (such as fidgeting, speaking out of turn, or restlessness) or by symptoms of all three. Examples include, without limitation, Attention-Deficit Disorder (ADD) and attention deficit hyperactivity disorder (ADHD).

As used herein, the term “autistic spectrum disorder” or “ASD” refers to autism and similar disorders. Examples of ASD include disorders listed in the Diagnostic and Statistical Manual of Mental Disorders (DSM-IV). Examples include, without limitation, autistic disorder, Asperger's disorder, pervasive developmental disorder, childhood disintegrative disorder, and Rett's disorder. Known ASD diagnostic screenings methods include, without limitation: Modified Checklist for Autism in Toddlers (M-CHAT), the Early Screening of Autistic Traits Questionnaire, and the First Year Inventory; the M-CHAT and its predecessor CHAT on children aged 18-30 months, Autism Diagnostic Interview (ADI), Autism Diagnostic Interview-Revised (ADI-R), the Autism Diagnostic Observation Schedule (ADOS)The Childhood Autism Rating Scale (CARS), and combinations thereof. Known symptoms, impairments, or behaviors associated with ASD include without limitation: impairment in social interaction, impairment in social development, impairment with communication, behavior problems, repetitive behavior, stereotypy, compulsive behavior, sameness, ritualistic behavior, restricted behavior, self-injury, unusual response to sensory stimuli, impairment in emotion, problems with emotional attachment, impaired communication, and combinations thereof.

The term “treat” or “treatment”, as used herein, refers to any type of treatment that imparts a benefit to a patient afflicted with a disease, including improvement in the condition of the patient (e.g., in one or more symptoms), delay in the progression of the condition, etc.

One embodiment of the present invention relates to a method for the diagnosis and/or evaluation and/or treatment of a disorder in a subject comprising placing said subject in proximity to a gaze contingent system, displaying a battery of tasks on the display of said system; obtaining data from the subject's gaze position and communicating the data to the processing component; subject's gaze position; and analyzing the data obtained from the subject's gaze response to said tasks.

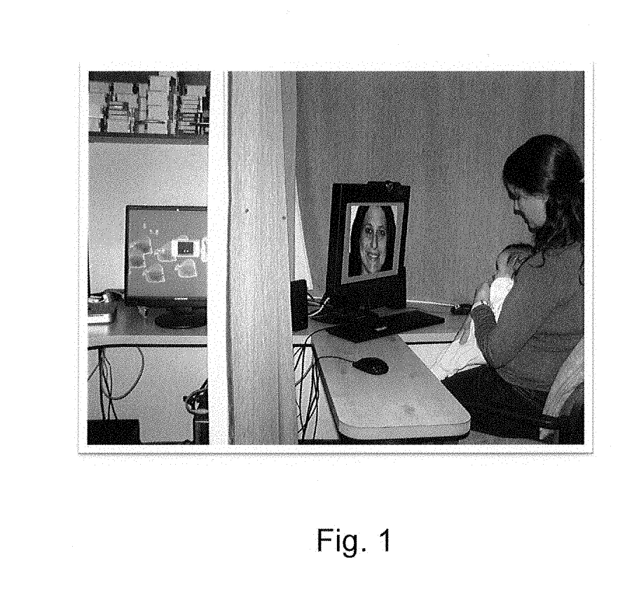

FIG. 1 shows one embodiment of the method of the invention, where an infant is placed in front of a display (screen) of the gaze contingent system of the invention, on which a task is being displayed (a clip or image). The eye tracker component, which is located below the display, tracks the infant's gaze position and communicates the data obtained to the processing component (on the left). The processing component changes the matter displayed and/or initiates and discontinues activities displayed on the display as a function of the infant's gaze.

In some embodiments, the battery of tasks comprises at least one social and visual attention task. According to some embodiments the social and visual attention task comprises images and clips which are displayed on the display.

In some embodiments, the method of diagnosis further comprises comparing the data obtained from the subject's gaze response with gaze patterns of healthy subjects.

The tasks are composed from various still and motion stimuli that are operated and terminated in response to the fixation of the infant/child. The stimuli are adjusted to fit the infant/child world of content and thus contain social content, non-social objects, stimuli with semantic value and low visual processing stimuli (vary on their contrast, shape, color and motion levels). The gaze contingency method allows an interactive intuitive and realistic use.

According to some embodiments, for example, an arousal task tests the subject preference for high repetitive arousing stimuli over low arousing more meaningful stimuli. The task comprises two images displayed side by side. Fixating on each of the images activates a clip with different spatial and temporal features. One side presents a high rate repetitive clip with simple details and high contrast. On the other side a slower rate more complex clip with lower contrast may be activated. A move of the subject's gaze from one image to the other terminates the clip and operates the new fixated one at the subject's will.

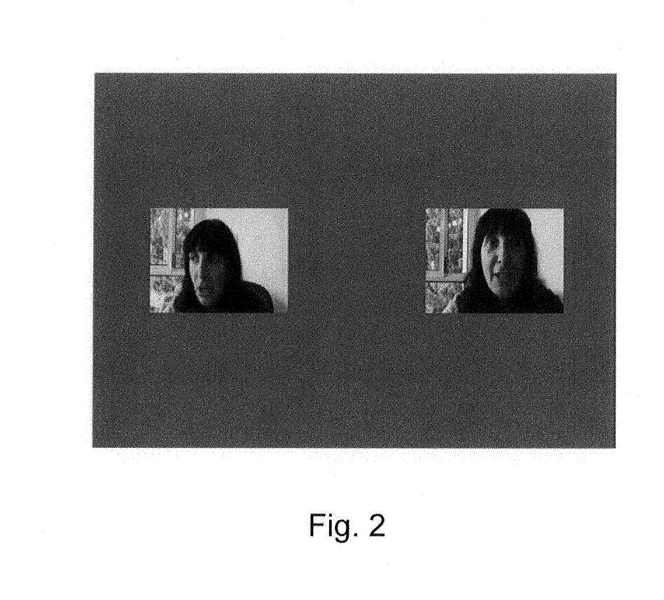

According to some embodiments, for example, a social task may test the subject's preference for direct eye gaze. The task comprises two similar images of the same woman displayed side by side on the screen, having different eye gaze directions. In one image, her eye gaze is directed at the subject while in the other, her gaze is directed away from the subject throughout the clip (FIG. 2). Fixating on each of the images activates a clip of the woman depending on the side of the screen that the subject fixates on. A move of the subject's gaze from one image to the other terminates the clip and operates the new fixated one. In this task the subject may switch his gaze between the images of the direct and indirect gaze positions on the screen.

In some embodiments, the gaze contingent system used in the method comprises a display, an eye tracker component and a processing component operatively connected to the display and eye tracker components. The processing component comprises circuitry adapted to initiate and to discontinue activities displayed on the display as a result of input received from the eye tracker component. In some embodiments, the circuitry associated with the processing component is configured to, change the image or sequence of images displayed on the display as a function of the subject's gaze position.

In some embodiments, the method of treatment comprises displaying a battery of tasks on the display, obtaining data from the subject's gaze position and communicating the data to the processing component, changing the display as a function of the subject's gaze position, analyzing the data obtained, and modifying the task displayed on the display for encouraging a social behavior.

In some embodiments, the battery of tasks comprises social and visual attention tasks that are individually-tailored to the subject's pace and capabilities. Task stimuli characteristics continually change according to his/her responses. This method enables waiting for the subject's response and making adjustments of the stimuli content and level accordingly. This method results in improving the response patterns of the subject and moderates the frequency and severity of their symptoms. The length of the tasks is also adjusted according to the attention span of the subject.

Another embodiment of the present invention relates to a gaze contingent system as discussed above, for use in treating a social communication disorder or an attention disorder in a subject.

In some embodiments of the present invention, the subject diagnosed/treated is a child, in some embodiments the subjects is an infant, who may be in some cases less than 2 years old and even a few months old.

According to some embodiments of the invention the subject is at risk or diagnosed with a social communication disorder or an attention disorder.

In some embodiments of the invention the social communication disorder or the attention disorder is associated with autism spectrum disorder (ASD), specific language impairment (SLI), learning disabilities (LD), language learning disabilities (LLD), intellectual disabilities (ID), developmental disabilities (DD), attention deficit hyperactivity disorder (ADHD), attention deficit disorder (ADD) and traumatic brain injury (TBI).

Another aspect of the present invention relates to an interactive gaze contingent system for evaluating and/or diagnosing and/or treating a social communication disorder or an attention disorder in a subject, comprising: a display, an eye tracker component and a processing component. The processing component is operatively connected to the display and eye tracker component. The processing component comprises circuitry adapted to initiate and to discontinue activities displayed on the display as a result of input received from the eye tracker component.

In some embodiments, the eye tracker is configured to obtain data from the subject's gaze position and to communicate it to the processing component. The processing component is configured to, based on the data obtained, change the matter displayed on the display as a function of the subject's gaze position.

In some embodiments, the circuitry associated with the processing component is configured to, change the image or sequence of images displayed on the display as a function of the subject's gaze position.

In one embodiment, the processing component is configured to run a battery of tasks. According to another embodiment, the battery of tasks comprises at least one social and visual attention task.

In yet another embodiment of the invention the social and visual attention task comprises images and clips that are displayed on the display and are manipulated by the subject's gaze using the gaze contingent methodology that includes an eye tracker that is operating a program kit.

### EXAMPLES

### Example 1

The aim of the study was to investigate differences in visual social preferences among two groups of 9 months, high-risk infants: infants born preterm and siblings of children diagnosed with ASD, compared to a low-risk group of infants. The study focused on one of the social aspects found impaired among individuals with ASD, namely, direct gaze preference. The behavior was measured by the infant's regulation of gaze to stimuli with socio-communicative value: social stimuli that involve opportunities for gaze contact.

Methods

Participants

The study cohort (N=114) comprised of 9 months old infants corrected age (mean=9.5, sd=0.92).

Recruitment of infants—Infants (N=61) born preterm at “Sheba” hospital were recruited for this study during the time of hospitalization (preterm group). Siblings of children diagnosed with ASD (N=18) were recruited using advertising through the Israeli Voluntary Association for ASD children (ALUT). Healthy term controls were recruited using word of mouth and parental internet sites (N=35). Exclusion criteria were serious complications after birth (like cerebral hemorrhage), hearing or vision deficits. One infant had serious vision difficulty and was therefore excluded from the analysis.

All families were recruited from the main urban county of Israel. Parents signed an informed consent letter to this study when recruited.

Procedure

Eye tracking assessment

To ensure a continuous recording of the data, the participants were tested individually in a comfortably illuminated (30 lux), quiet inner room, enclosed within a grey curtained chamber, isolated from the experimenter who was monitoring their gaze behavior throughout the trial on a separate display.

Participants were positioned in front of a computer at an approximate 60 cm distance from the screen. Infants sat on the laps of their caregiver or in a baby high chair with the caregiver standing behind them. Mothers were instructed to turn their head away from the screen or to keep their eyes shut during the stimuli presentation, so that their gaze wouldn't interfere throughout the task. A Tobii© 1750 eye tracker that tracks both eyes at a rated accuracy of 0.5 degrees at a rate of 50 _{HZ} was used. The task was programed specifically for the aims of the current study and operated using the EPRIME© program. The images and clip stimuli of the tasks were presented on a display with a resolution of 1280*1024 pixels. Prior to data collection, an eye calibration was performed using a 5-point system.

Gaze Contingent Paradigm

Gaze contingency was enabled by using gaze coordinates as an online input to the running procedure so that the display of the task continually changes according to the fixations of the subject. This technology allowed creating a side by preference task, where the infant's gaze operated and terminated clips containing social content at different complexity levels.

Computer Task Description

A social task was designed to observe the preference of direct eyegaze. The task had two similar images of the same woman, unfamiliar to the subject, having different eye gaze directions. In one image, her eye gaze was directed at the infant while in the other, her gaze was directed away from the infant throughout the clip (FIG. 2). Fixating on each of the images activated a 20 sec long clip of the woman depending on the side of the screen that the infant fixated on. A move of the infant's gaze from one image to the other terminated the clip and operated the new fixated one. The task had two blocks in order to switch between the positions of the direct and indirect gaze positions on the screen.

Throughout the task, two areas of interest (AOI) were defined for coding purposes, each covering the full area on the screen where the clip was presented. Each AOI was at the size of 25% of the X axis*25% of the Y axis. The left AOI was centered at 25% on the X axis and 50% on the y axis and the right one is 75% on the x axis and 50% on the Y axis.

Questionnaire for Classifying Concern (High Risk) vs. Non-Concern (Low Risk) Groups

To explore parents' report on the infant social behavioral markers, the parents were also given a screening questionnaire to fill. This questionnaire, The Communication and Symbolic Behavior Scales Developmental Profile (CSBS DP), is a parental report that is designed to identify children who are at risk of developing communication and/or social impairments [Pierce, K., et al., Detecting, Studying, and Treating Autism Early: The One-Year Well-Baby Check-Up Approach. The Journal of Pediatrics, 2011. 159(3): p. 458-465.e6], includes 24 questions divided into 3 categories: communication, expressive speech and symbolic. The total score of the questionnaire was used to classify the cohort to concern (high risk) vs. non-concern (low risk) groups.

Exclusion Criteria

Exclusion criteria that were implemented following data collection:

“Sticky Gaze” Infants

Some infants locked their gaze onto one side of the screen throughout most of the procedure, although the stimuli were equally presented on both sides of the screen. This “sticky gaze” pattern precluded the infant from presenting a preference of one item over another, and thus could not be included in the analysis.

The Exclusion Criterion.

The calculation of this exclusion criterion was as follows:

$$\begin{matrix}M = abs ( {Σ}_{i = 1}^{N} if ( ( Total time {left}^{( i )} - Total time {Right}^{( i )} ) , then 1 , else 0 ) ) . & 1 \\ R = \frac{{Σ}_{i = 1}^{N} Total time {left}^{( i )}}{{Σ}_{i = 1}^{N} Total time {Right}^{( i )}} . & 2\end{matrix}$$

Where N is the number of blocks in the task (equals 4)

Exclusion criterion=if (M==4&& (R>5 or R<0.2), then 1, else 0)

Participants having an Exclusion criterion==1, were excluded from the analysis.

These infants (N=9, 3 from preterm group (5%), 2 from siblings group (11%) and 4 from control group (11%)) were excluded from the analysis because their “sticky gaze” pattern may interfere with their preferences. After this exclusion factor, together with the infant that was excluded due to a severe vision impairment the cohort included a total of 104 infants.

Exclusion Criteria for Missing Gaze Data Blocks

The exclusion criterion for blocks with missing data was total fixation time that was shorter than 10% of the block presented time. 9.6% of total blocks were excluded, more specifically, 11.5% of directed gaze task's blocks, were excluded due to this exclusion criterion.

Finally, four participants from the control group received a concern result aftercoding the CBSC-DP screening questionnaire. They were excluded in order to create a purer low risk group. The exclusion of this criteria, resulted in a sample of N=100 for the direct gaze Vs. averted gaze preference analysis.

TABLE 1
Demographic statistics of participants as a function of risk
group. Standard deviation in brackets

| | Control | Preterms | Siblings |
|---|---|---|---|
| N | 27 | 57 | 16 |
| Gender | 40.74% girls | 47.37% girls | 43.75% girls |
| Gestation age | 39.46 (±1.3) | 30.63 (±2.96) | 38.8 (±1.16) |
| (weeks) | | | |
| Wight at birth | 3341 (±447) | 1390 (±578) | 3171 (±605) |
| (Grams) | | | |
| Age at visit at lab | 9.5 (±0.81) | 9.57 (±0.97) | 9.36 (±0.79) |
| (in months) | | | |

Data Analysis and Statistics

Data was recorded using Eprime© and analyzed using Matlab©. The analysis targets all fixations during the entire task using the velocity threshold identification (I-VT) algorithm [Salvucci, D. D. and J. H. Goldberg, Identifying fixations and saccades in eye-tracking protocols, in Proceedings of the 2000 symposium on Eye tracking research & applications. 2000, ACM: Palm Beach Gardens, Fla., USA. p. 71-78]. This is a velocity-based algorithm, where the velocity between each pair of sequential points is calculated and gaze points are classified as being either a fixation (below threshold) or a saccade (above threshold). The velocity threshold was set to be 150 degree/sec. Merging two sequential fixations was based on a max time gap of 80 ms as well as a max angle of 0.5 degree between the two sequential fixations. Very short fixations beneath 60 ms were eliminated [Olsen, A., The Tobii I-VT Fixation Filter. 2012].

After the fixations were defined, each infant's Total fixation time at each AOI was calculated. Two Repeated measures ANOVAs were performed for each task separately, with total time of fixation duration at each clip as a within subjects factor. The between subject factor was the classification into groups (siblings, preterm and control for one analysis and concern vs. non-concern for the other analysis). Further analysis used the same within-subjects factor with the control and preterm groups only as between subject factor in order to further examine the interaction between these two groups and direct gaze preference

The corrected age of the infant at the time of the test was entered as covariates at all analyses.

Results

High Risk Vs. Low Risk Groups Differences in the Direct Gaze Preference

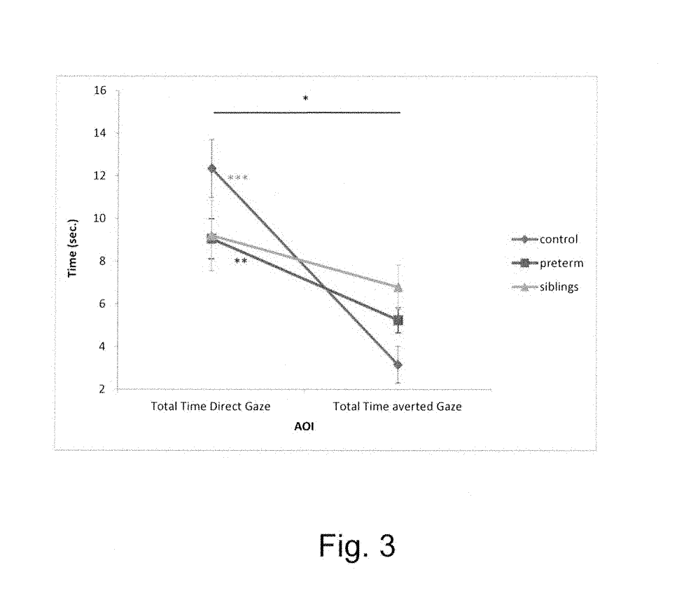

In order to explore High risk Vs. Low risk groups differences in the direct gaze preference, analysis of variance with repeated measures was conducted exploring direct gaze preference over averted gaze preference as a function of risk group 3 levels, preterms (N=49), and siblings (N=16) and a low risk group (N=23). The within subject factor was total fixation time at AOI's, e.g. directed gaze AOI Vs. averted gaze AOI. Results indicated an interaction between total fixation at AOI's and group (F=4.466, p=0.014, η²==0.096, FIG. 3), There was no main effect for risk group indicating no differences in total fixation time on task between groups. To understand the source of the interaction, ANOVA was conducted showing a preference for direct gaze over averted gaze for the control group (F=29.938, p<0.001, η²=0.263) and preterm group (F=10.93, p=0.001, η²=0.115) but not for the siblings group (F=1.426, p=0.236, η²=0.017). Further univariate tests indicated differences between the groups in total fixation time at the averted gaze cue (F=3.876, p=0.025, η²=0.084 and that the control group had more pronounced preference for direct gaze over averted gaze than both at risk groups. Nevertheless, the preterm group still presented preference (lower than the controls) for direct gaze over averted gaze, where the siblings did not present a significant preference.

Further analysis was performed in order to examine the interaction between the control and the preterm group separately using Repeated measures ANOVA. Total fixation time at directed gaze AOI Vs. averted gaze AOI was the within subject factor and the risk group (control Vs. preterm) as the between subject factor. Results indicated, on an Interaction between gaze preference and group (F=9.594, p=0.003, η²=0.122). More specifically, higher preference for direct gaze over averted gaze in the low risk group comparing to the preterm high risk.

Univariate tests indicated differences between the two groups in total fixation time at both directed gaze cue (F=5.685, p=0.02, η²=0.076) and the averted gaze cue (F=4.269, p=0.043, η²=0.058). These Results emphasize that although both groups showed preference for direct gaze, the control group preference for direct gaze is more significant.

FIG. 3 shows mean total fixation duration at Direct gaze AOI (left side) and at averted gaze AOI (Right side). Marked with [symbol] are infants from the control group, [symbol] are infants from the preterm group and with [symbol] are infants from the siblings group. The straight black line represents the interaction between total fixation duration at AOI's and group.

Concern Vs. Non Concern Groups Differ in the Direct Gaze Preference

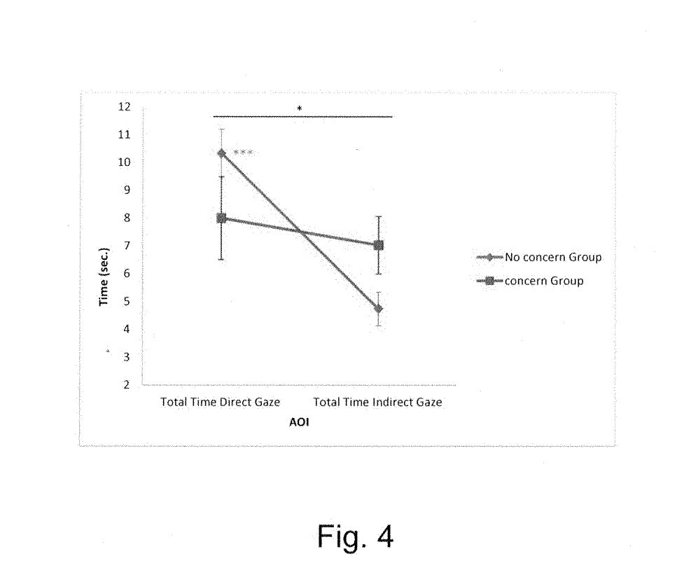

In order to test differences between High risk Vs. Low developmental concern groups in the direct gaze preference task, repeated measurers ANOVA was performed with total fixation time at AOI's (directed gaze Vs. averted gaze) as the within subject factor, and concern grouping as the between subject factors. The results showed an interaction between gaze preference and concern grouping (F=4.117, p=0.046, η²=0.057, see FIG. 4) suggesting that subjects at the concern group (N=18, 14.8% from control group 43.75% from siblings group and 12.3% from preterm group), do not show significant preference for direct gaze over averted gaze, whereas those without no concern (N=53) showed a preference for direct gaze (F=23.865, p<0.001, η²=0.26). There was no main effect of concern grouping, indicating that there was no difference between groups in total fixations time at the task. The interaction results purely from the direct gaze preference differences between the groups. These results show that while non-concern infants show a very significant preference for direct gaze over averted gaze; infants screened with developmental risk for social deficits at 9 months, show no preference in the direct gaze preference task.

FIG. 4 shows mean total fixation duration at Direct gaze AOI and at averted gaze AOI. Marked with [symbol] are infants from the non-concern group and with [symbol] are infants from the concern group. The straight black line represent the interaction between total fixation duration at AOI's and group.

### Example 2

The aim of the study was to investigate differences in non-social visual preferences among two groups of 9 months old high-risk infants: infants born preterm and siblings of children diagnosed with ASD, compared to a low-risk group of infants. The study focused on preference for non-social stimuli distinguished by their spatial and temporal characteristics. The “simple” stimulus was a clip with high contrast, clear and simple shape and few colors (red, white, black) and was presented in high motion rate. The “complex” stimulus was a clip with lower contrast, more detailed in manners of shape and colors and was presented in slow motion rate.

Methods

As in Example 1 above.

Results

High-Risk Vs. Low-Risk Groups Differences in the Stimuli Preference

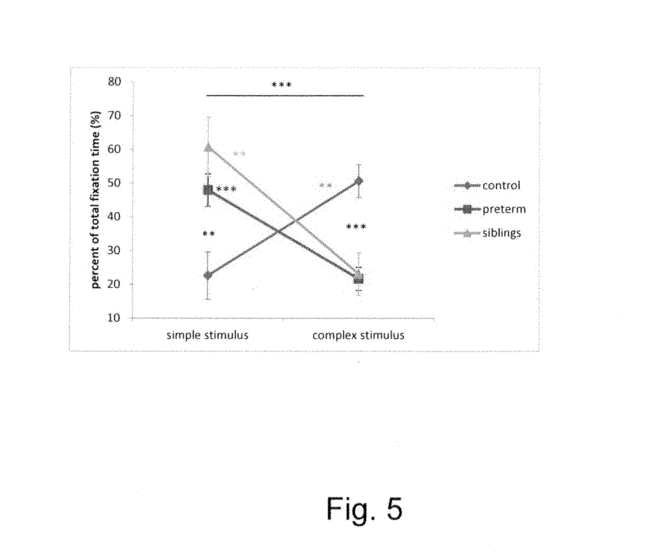

Analysis of variance with repeated measures showed marked differences as a function of group (see FIG. 5).

The ANOVA with the risk group as an independent factor with the corrected age as a covariate showed an interaction between total fixation at AOI's and group (F=11.306, p<0.0001, η²==0.203). In order to understand the source of the interaction, ANOVA was conducted showing a preference for the complex stimulus over the simple stimulus for the control group (F=7.291, p=0.008, η²=0.076) and an opposite preference for the simple over the complex stimulus for the siblings group (F=8.196, p=0.005, η²=0.084) and the same for the preterm group (F=13.357, p<0.001, η²=0.130). Further univariate tests indicated differences between the groups in total fixation time at the complex stimuli (F=12.258, p<0.0001, η²=0.216) and at the simple stimuli (F=6.836, p=0.002, η²=0.133).

FIG. 5 shows mean total fixation duration at the simple stimulus (left side) Vs. the complex stimulus (right side). Marked with [symbol] are infants from the control group, [symbol] are infants from the preterm group and with [symbol] are infants from the siblings group. The straight black line represents the interaction between total fixation duration at AOI's and group.

Siblings Group Vs. Preterm and Control Groups differences in Fixation Duration During Task-Differentiating Between the High Risk Groups

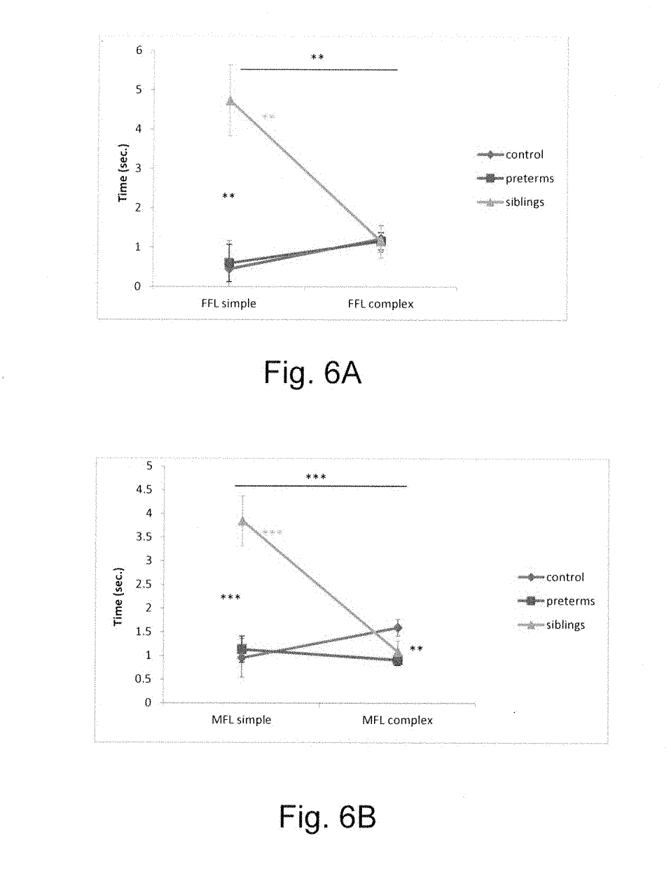

In order to explore High-risk Vs. Low-risk groups differences in the first fixation duration (FFL) to different stimuli, analysis of variance with repeated measures was conducted exploring FFL during the observation of simple Vs. complex stimuli as a function of risk group 3 levels, control (N=23), Preterms (N=53) and siblings (N=15). The within-subject factor was FFL at the simple Vs. complex AOI's. The between-subject factor was the risk group and corrected age as a covariate. Results indicated an interaction between FFL at simple Vs. complex AOI's and risk group (F=7.481, p=0.001, η²=0.147, FIG. 6(a)). In order to understand the source of the interaction, ANOVA was conducted showing no differences in FFL between AOI's for the control group and the preterm group. As for the siblings group, there was a great difference in FFL between the two AOI's (F=12.986, p=0.001, η²=0.130). Further univariate tests indicated differences between the groups in FFL at the simple stimuli (F=8.949, p<0.001, η²=0.171) but not the complex one. These results indicate a dramatic increase in first fixation duration when viewing the simple stimulus compared to complex stimulus for the siblings group, while the two other groups does not show any difference in FFL between the two clips.

In order to explore whether this pattern remains for the mean fixation time (MFL), a similar analysis of variance with repeated measures was conducted, only with testing MFL (instead of FFL) during the observation of the simple Vs. complex stimuli as a function of risk group 3 levels, control (N=24), Preterms (N=52) and siblings (N=15). Results indicated an interaction between MFL at AOI's and risk group (F=13.342, p<0.00001, η²=0.235, FIG. 6(b)). In order to understand the source of the interaction, ANOVA was conducted showing no differences in MFL between AOI's for the control group and the preterm group. As for the siblings group, there was a great difference in MFL between the two AOI's (F=27.36, p=0.000001, η²=0.239). Further univariate tests indicated differences between the groups in MFL at the simple stimuli (F=11.692, p<0.0001, η²=0.212) and for the complex one (F=5.295 p=0.007, η²=0.09. These results indicate that here similarly to the FFL analysis a dramatic increase in mean fixation duration occurs when viewing the simple stimulus compared to the complex stimulus for the siblings group, while the two other groups do not show any difference in MFL between the two clips.

FIG. 6A shows mean first fixation time (FFL) and FIG. 6B shows mean fixation time (MFL) at the simple stimulus (left side) and at complex stimulus (right side). Marked with [symbol] are infants from the control group, [symbol] are infants from the preterm group and with [symbol] are infants from the siblings group. The straight black line represents the interaction between fixation duration at AOI's and group.

The above illustrates the usefulness of the invention and its efficacy in obtaining the desired results.

Although embodiments of the invention have been described by way of illustration, it will be understood that the invention may be carried out with many variations, modifications, and adaptations, without exceeding the scope of the claims.

1. An interactive gaze contingent system for evaluating and/or diagnosing and/or treating a social communication disorder or an attention disorder in a subject, comprising:
a display;
an eye tracker component; and
a processing component operatively connected to the display and eye tracker component, wherein said processing component comprises circuitry adapted to initiate and to discontinue activities displayed on said display as a result of input received from said eye tracker component.

2. The system of claim 1, wherein the eye tracker is configured to obtain data from the subject's gaze position and to communicate it to the processing component.

3. The system of claim 1, wherein the circuitry associated with the processing component is configured to, change the image or sequence of images displayed on the display as a function of the subject's gaze position.

4. The system of any one of claim 1, wherein the processing component is configured to run a battery of tasks.

5. The system of claim 4, wherein the battery of tasks comprises at least one social and visual attention task.

6. The system of claim 5, wherein the social and visual attention task comprises images and clips that are displayed on the display.

7. A method for the diagnosis and/or evaluation and/or treatment of a disorder in a subject comprising placing said subject in proximity to a gaze contingent system, displaying a battery of tasks on the display of said system; obtaining data from the subject's gaze position and communicating the data to the processing component; changing the matter displayed on the display as a function of the subject's gaze position; and analyzing the data obtained from the subject's gaze response to said tasks.

8. The method of claim 7, further comprising comparing data from the subject's gaze response with gaze patterns of healthy subjects.

9. A method according to claim 7, for the treatment of a disorder in a subject, further comprising modifying the task displayed on the display for encouraging a social behavior.

10. The method of claim 7, wherein the battery of tasks comprises at least one social and visual attention task.

11. The method of claim 10, wherein the social and visual attention task comprises images and clips that are displayed on the display.

12. The method of claim 7, wherein the disorder is a social communication disorder or an attention disorder.

13. The method of claim 12, wherein the social communication disorder or attention disorder is associated with autism spectrum disorder (ASD), specific language impairment (SLI), learning disabilities (LD), language learning disabilities (LLD), intellectual disabilities (ID), developmental disabilities (DD), attention deficit hyperactivity disorder (ADHD), attention deficit disorder (ADD) and traumatic brain injury (TBI).

14. The method of claim 7, wherein the subject is an infant.

15. The method of claim 14, wherein the subject is less than 2 years old.

16. The method of claim 15, wherein, the subject is less than 1 year old.

17. The method of claim 7, wherein, the subject is a child.
